SEQUENCE LISTING (1 sequence)

SEQ ID NO: 1 - protein, 512 aa, Artificial
  Synthetic construct
MQTSPTGILPTTSNSISTSEMTWKSSFPEFARYTTPEDTTPEPGEDPRVTRHSGQNHLKEMAISVLEARACAAAGQTVSVVALHDDMENQPLIGIQSTAIPEVATRMQSFGMKIVGYDPIISPEVAIIQVSPKDIQLTIFPSKSVKEGDTVKASKKGMWSEGNSSHTIRDLKYTIETSIPSVSNALNWKEFSFIQSTLGYVLRTAAYVNAIEKIFKVYNEAGVTFTSWIHCWKYLSVQSQLFRGSSLLFRRSNFTVDCSKAGNDMLLVGVHGPRTPALGSLALMIWLMTTPHSHETEQKRLLPGFKGVKGHSGIDGLKGQPGAQGVAVQKLNLQNLVILQAPENLTLSNLSESDRNKESSDQTSVNMNGLENKISYLLPFYPPDEALEIGLELNSSALPPTILPQAPSGPSYATYLQPAQAQMLTPKPLRRNNSYTSYIMAICGMPLDSFRVIQTSKYYMRDVIAIESAWLLELAPHIHRAGGLFVADAIQVGFGRIGKHFGYPYDVPDYAS

(19) United States
(12) Patent Application Publication
(10) Pub. No.: US 2021/0154277 A1
(43) Pub. Date: May 27, 2021

(54) IMMUNOTHERAPEUTIC COMBINATION FOR TREATING CANCER

(71) Applicants: Nektar Therapeutics, San Francisco, CA (US); Nouscom AG, Basel (CH)

(72) Inventors: Jonathan ZALEVSKY, Berkeley, CA (US); Alfredo NICOSIA, Naples (IT); Elisa SCARSELLI, Rome (IT); Anna Morena D'Alise, Rome (IT)

(21) Appl. No.: 16/762,397

(22) PCT Filed: Nov. 6, 2018
(86) PCT No.: PCT/US2018/059498

§ 371 Date: May 7, 2020

Related U.S. Application Data
(60) Provisional application No. 62/582,774, filed on Nov. 7, 2017.

(51) Int. Cl. A61K 39/00; A61P 35/00; C07K 16/28; A61K 38/20

(52) U.S. Cl. CPC A61K 39/0011; A61P 35/00; A61K 2039/51; A61K 38/2013; C07K 16/2818

(57) ABSTRACT

Provided herein are methods and compositions for treating a subject having cancer by administering to the subject a neoantigen-based vaccine composition and a long acting, IL-2RPβ-selective agonist composition comprised of compounds of Formula (I), and optionally, an anti-PD-1 antibody.

### CROSS REFERENCE TO RELATED APPLICATIONS

This application claims the benefit of priority under 35 U.S.C. 119(e) to U.S. Provisional Patent Application No. 62/582,774, filed on Nov. 7, 2017, the disclosure of which is incorporated herein by reference in its entirety.

### FIELD

The instant application relates to (among other things) the field of immunotherapy, and in a particular aspect, cancer immunotherapy, and involves the treatment of an individual having cancer by administering to the individual a neoantigenic cancer vaccine composition in combination with a CD-122-biased cytokine agonist, optionally further combined with an anti-PD-1 antibody.

### BACKGROUND

Therapeutic cancer vaccines represent a class of substances that work by stimulating or restoring a subject's immune system's ability to fight infections and disease. Therapeutic vaccines, as opposed to preventative or prophylactic vaccines, are used to treat an existing cancer by boosting the body's natural immune response against the cancer and represent a type of immunotherapy. Cancer treatment vaccines are designed to activate cytotoxic T cells and direct them to recognize and act against specific types of cancer or to induce production of antibodies that bind to molecules on the surface of cancer cells. However, producing effective therapeutic vaccines has proven to be a challenging endeavor, because the vaccine intervention must combat the body's immune system that is restrained by mechanisms that work to sustain the cancer. To be effective, a therapeutic cancer vaccine must not only stimulate a specific immune response against the intended target, but must also be powerful enough to overcome the barriers that cancer cells utilize to protect themselves from attack by killer T cells. Over the last several years there have been substantial efforts in developing therapeutic vaccines encompassing various platforms, however, only one vaccine, Provenge® (sipuleucal-T, an autologous vaccine), has received FDA approval to date. Therapeutic vaccines have been evaluated, for example, in patients with breast cancer, lung cancer, melanoma, pancreatic cancer, colorectal cancer, and renal cancer (Melero, I., et al., Nat Rev Clin Oncol, 2014, 11 (9), 509-524).

To improve immunization anticancer strategies, substances such as adjuvants can be added to vaccines to boost their ability to induce potent anticancer immune responses, although improved responses can often be partial and/or transient. Adjuvants for cancer vaccines can come from a variety of sources, such as bacteria, substances produced by bacteria, proteins, and synthetic or natural cytokines. Various substances including cytokines have been investigated for enhancing vaccine-induced antitumor activity. While some cytokines appear to function as effective adjuvants, others have been found to be surprisingly ineffective in modulating vaccine effectiveness. Cytokines used in cancer treatment vaccines include, for example, IL-2, interferon-alpha, and granulocyte-macrophage colony stimulating factor (GM-CSF).

Although there have been substantial efforts in developing therapeutic vaccines encompassing various platforms to date, there remains a need to identify and provide new and more effective immunotherapeutic vaccines and related treatment regimes. Thus, the present disclosure seeks to address this and other needs.

### SUMMARY

In a first aspect, provided herein is a method for treating a subject having cancer, by administering to the subject, (i) a vaccine composition comprising a neoantigen-based vaccine composition comprising a first vector comprising a nucleic acid construct encoding multiple (i.e., more than one) immunogenic polypeptide fragments, each of a protein mutated in cancer cells, wherein each immunogenic polypeptide fragment comprises one or more mutated amino acids flanked by a variable number of wild type amino acids from the original protein, joined head-to-tail to form an immunogenic polypeptide, (referred to herein as “NBV composition”) and (ii) a long acting, IL-2Rβ-selective agonist composition comprising compounds of Formula (I),

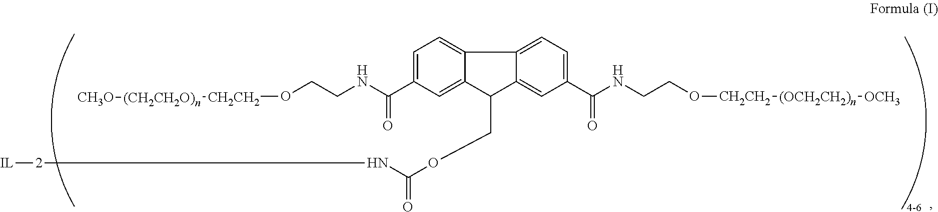

wherein IL-2 is an interleukin-2, “—NH-IL-2” represents an amino group of the interleukin-2, and each integer (n) has a value from about 3-4000, or from about 200-300, or pharmaceutically acceptable salts thereof, (referred to herein as (2,7-(bis-methoxyPEG-carboxyamide)(9H-fluorene-9-yl)methyl N-carbamate)₄₋₆interleukin-2 or “RSLAIL-2”), and optionally, (iii) an anti-Programmed Death-1 (PD-1) antibody (e.g., nivolumab).

In a second aspect, provided herein is a method of enhancing the therapeutic efficacy of the NBV composition, by administering to a subject having cancer the NBV composition described above, and an IL-2Rβ-activating amount of RSLAIL-2, and optionally, an anti-PD-1 antibody, i.e., nivolumab, wherein RSLAIL-2, optionally administered in combination with an anti-PD-1 antibody such as nivolumab, is effective to improve the subject's immune response to the NBV composition.

In one or more embodiments wherein the cancer comprises a cancerous tumor, numbers of NBV composition-induced T-cells in the tumor, such as CD4 and CD8 T cells reactive to each of the vaccine-encoded neoantigens, are increased over numbers of such T-cells determined upon administration of the NBV composition alone.

In one or more embodiments related to the NBV composition, the lengths of each of the immunogenic polypeptide fragments can vary.

In some further embodiments related to the NBV composition, the nucleic acid construct may encode an immunogenic polypeptide comprising at least one immunogenic polypeptide fragment, preferably at least 4 immunogenic polypeptide fragments, and more preferably more than 19 immunogenic polypeptide fragments, each of a protein mutated in cancer cells, joined head-to-tail. An illustrative mouse cancer neoantigenic polypeptide comprising multiple immunogenic polypeptide fragments used in the supporting examples is provided in SEQ ID NO:1 (FIG. 1).

In some further embodiments, the vector is an adenoviral vector, such as for example, a nonhuman great ape-derived adenoviral (GAd) vector.

In yet some additional embodiments, the NBV composition further comprises a second vector comprising a nucleic acid construct encoding multiple (i.e., more than one) immunogenic polypeptide fragments, each of a protein mutated in cancer cells, wherein each immunogenic polypeptide fragment comprises one or more mutated amino acids flanked by a variable number of wild type amino acids from the original protein, joined head-to-tail to form an immunogenic polypeptide, and at least one epitope of the immunogenic polypeptide encoded by the first vector is immunologically identical to at least one epitope of the immunogenic polypeptide encoded by the nucleic acid construct of the second vector, for use in a prime-boost vaccination regimen. In some related embodiments, the second vector is a modified vaccinia Ankara (MVA) vector.

In yet some further embodiments related to RSLAIL-2, the long acting, IL-2Rβ-selective agonist composition contains no more than about 10 percent (molar) of compounds encompassed by the following formula:

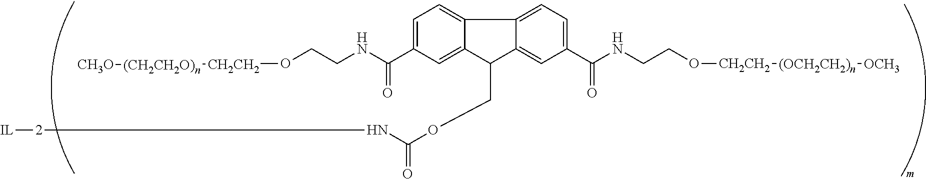

wherein (m) is an integer selected from the group consisting of 1, 2, 3, 7 and >7, or pharmaceutically acceptable salts thereof, and each integer (n) has a value from about 200-300.

In some additional embodiments related to RSLAIL-2, each of the “m” branched polyethylene glycol moieties of Formula (I) has a weight average molecular weight of about 20,000 daltons.

In some further embodiments of the method, the cancer is selected from, for example, the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease and adrenocortical cancer.

In yet a further, third aspect, provided herein is a method of treating cancer in a subject, comprising administering to the subject the NBV composition, an IL-2Rβ-activating amount of RSLAIL-2, and optionally, an anti-PD-1 antibody, wherein when evaluated in a mouse model of the cancer, treatment is effective to reduce the size of a cancerous tumor by at least about 30% (partial response), or by at least about 40%, or by at least about 50%, or by at least about 60%, or by at least about 70%, or at least about 80%, or at least about 90%, or to result in complete tumor regression, when compared to the size of the tumor prior to the administering. In some particular embodiments, the method when evaluated in a mouse model of the cancer, is effective to reduce the size of a cancerous tumor by at least about 30% (a partial response).

By way of clarity, with regard to the sequence of administering, the NBV composition and RSLAIL-2, and optionally an anti-PD-1 antibody such as nivolumab, may be administered concurrently or sequentially, and in any order, and via the same and/or different routes of administration. Moreover, treatment may comprise a single cycle of therapy, or may comprise multiple cycles, where follow-on cycles may comprise administration of each of the NBV composition, RSLAIL-2, and an anti-PD-1 antibody, or may comprise administration of fewer than each of the foregoing therapeutic components, e.g., administration of the NBV composition and RSLAIL-2, or administration of the NBV composition and an anti-PD-1 antibody, or administration of RSLAIL-2 and an anti-PD-1 antibody, or administration of the NBV composition, or administration of RSAIL-2, or administration of an anti-PD-1.

For example, in some embodiments of the method, the NBV composition is administered to the subject prior to administering RSLAIL-2. In some particular embodiments, the NBV composition and RSLAIL-2 are both administered on day 1 of treatment. In yet some additional embodiments, the NBV composition is administered on day 1 of treatment and RSLAIL-2 is administered on a day greater than 5 days following vaccination (e.g., on day 6, 7, 8, 9, 10, 11, 12, 13, 14, 15, or greater, of treatment).

In yet some additional embodiments, the method comprises first administering the NBV composition for priming an immune response in the subject, and a subsequent administration of the same vaccine NBV composition or a NBV composition directed against the same disease for boosting the immune response in the subject. In some particular embodiments related to the foregoing, the subsequent administration for boosting the immune response of the subject is administered within 1 week, 2 weeks, 3 weeks or 4 weeks or more following the first administering.

In some embodiments, the dose of RSLAIL-2 ranges from about 0.0001 mg/kg to about 0.1 mg/kg body weight.

In yet some further embodiments, the method comprises administering to the subject an anti-PD-1 antibody, wherein the anti-PD-1 antibody is preferably nivolumab.

In some embodiments related to the foregoing, nivolumab may be administered (i) on the same day as the NBV composition, (ii) on the same day as RSLAIL-2, (iii) following administering of the NBV composition, (iv) following administering of RSLAIL-2, (v) following administering of the NBV composition but before administering of RSLAIL-2.

In yet some additional embodiments, the anti-PD-1 antibody, preferably nivolumab, is administered more than once over the course of therapy.

In some embodiments of the method, the NBV composition, RSLAIL-2, and nivolumab are administered as separate compositions.

In yet additional embodiments, the cancer comprises a cancerous tumor and the method is effective to reduce the size of the cancerous tumor when compared to the size of the tumor prior to the administering. Or in some more particular embodiments, cancer comprises a cancerous tumor and the method is effective to reduce the size of the cancerous tumor by at least about 30% (partial response), or by at least about 40%, or by at least about 50%, or by at least about 60%, or by at least about 70%, or at least about 80%, or at least about 90%, or to result in complete tumor regression, when compared to the size of the tumor prior to the administering. In yet some further embodiments of the method, the cancer comprises a cancerous tumor and the method is effective to result in complete tumor regression.

In some embodiments relating to any one or more of the foregoing aspects, when treating a solid cancerous tumor, the method is effective to result in a reduction in solid tumor size of at least about 25% when evaluated after 1 cycle of treatment.

In yet a further aspect, provided is a kit comprising the NBV composition, RSLAIL-2, and optionally, an anti-PD-1 antibody, i.e., nivolumab, accompanied by instructions for use in treating a subject having cancer.

In one or more embodiments of the kit, the NBV composition, RSLAIL-2 and optionally, an anti-PD-1 antibody such as nivolumab, are comprised in a single composition for administration to the subject, where the single composition optionally further comprises a pharmaceutically acceptable excipient.

In some alternative embodiments of the kit, the NBV composition, RSLAIL-2, and optionally, the anti-PD-1 antibody, i.e., nivolumab, are provided in separate containers, and the kit comprises instructions for administering each of the foregoing, i.e., the NBV composition, RSLAIL-2, and optionally, the anti-PD-1 antibody, e.g., nivolumab separately to the subject.

In some embodiments of the kit, both the NBV composition and RSLAIL-2 are in solid form. In yet a further embodiment, the anti-PD-1 antibody, i.e., nivolumab, when comprised in the kit, is in solid form. In one or more related embodiments, the NBV composition and RSLAIL-2, and optionally, the anti-PD-1 antibody, i.e., nivolumab, are each in a solid form suitable for reconstitution in an aqueous diluent.

In yet one or more further embodiments, each of the NBV composition, RSLAIL-2, and optionally, the anti-PD-1 antibody, i.e., nivolumab, are comprised within separate compositions each comprising a pharmaceutically acceptable excipient.

In yet some additional embodiments related to solid compositions, each of the NBV composition, the RSLAIL-2 solid composition, and the optional anti-PD-1 antibody, i.e., nivolumab, compositions contains less than 5 percent by weight water.

Additional aspects and embodiments are set forth in the following description and claims.

### BRIEF DESCRIPTION OF THE DRAWINGS

FIG. 1 provides the NOUS-020 insert sequence that corresponds to 20 neoantigens from the CT26 murine tumor cell line, as described in the Examples.

FIGS. 2A and 2B. As described in Example 4, FIGS. 2A and 2B illustrate the immunogenicity of the NOUS-020 vaccine. Analysis of T cell responses measured 3 weeks post immunization in naive mice by IFN-γ ELISpot on single mutated peptides is shown in FIG. 2A and on a pool of 20 peptides by intracellular cytokine staining in FIG. 2B (pool of peptides). Shown are the responses to the 5 immunogenic peptides (#3, 10, 17, 18, 19). ID epitopes correspond to position of the antigen in the construct where SFC refers to Spot Forming Cells. As shown, NOUS-020 GAd vaccine induces CD4 and CD8 T cells.

FIG. 3A provides a schematic of constructs showing the neontigens inducing the CD8 and CD4 response in the study described in Example 5. FIG. 3B provides an analysis of T cell responses measured post GAd/MVA immunization in naive mice by IFN-γ ELISpot on pool of 20 vaccine encoded neo-antigens.

FIGS. 4A-4F are plots of CT26 tumor growth in Balb/c mice receiving either no treatment, treatment with NOUS-020 GAd vaccine alone, treatment with RSLAIL-2 alone, or treatment with a combination of NOUS-020 GAd vaccine and RSLAIL-2 as described in Example 6. FIG. 4A provides results for the control group (untreated); FIG. 4B demonstrates volume of CT26 tumors in mice treated with GAd vaccine alone; FIGS. 4C and 4D demonstrate volume of CT26 tumors in mice treated with RSLAIL-2 (administered at either day 0 or 7, respectively) and concomitant at Day 0 (FIG. 4E) or sequential administration (FIG. 4F) of RSLAIL-2 and GAd, respectively.

FIG. 5A is a plot of tumor volume in individual mice with established tumors treated with RSAIL-2 alone; FIG. 5B is a plot of tumor volume in individual mice with established tumors treated with a combination of NOUS-020 vaccine and RSAIL-2 as described in Example 7. CR=complete response PR=partial response (>40% tumor shrinkage).

FIGS. 6A and 6B provide an analysis of immune response at day 54 measured in the spleen of mice responding to treatment with (i) RSLAIL-2 only, and (ii) NOUS-020 and RSLAIL-2, respectively, as described in Example 7. The T cell response against the pool of top 5 immunogenic neo-antigens and against the remaining 15 neoantigens encoded by the vaccine were quantified by ICS. Dashed and solid line represent a threshold for a positive response respectively for CD4 and CD8 T cells.

FIG. 7 is a bar graph illustrating percentage of mice with tumor shrinkage following treatment of established CT 26 tumors with (i) anti-PD-1 (clone RMP1-14), (ii) RSLAIL-2, (iii) anti-PD-1 and RSLAIL-2, (iv) RSLAIL-2 and NOUS-020, and (v) anti-PD-1 (clone RMP1-14), RSLAIL-2 and NOUS-020 as described in Example 8.

### DETAILED DESCRIPTION

The instant application relates to (among other things) the field of immunotherapy, and in a particular aspect, cancer immunotherapy, and involves the treatment of an individual having cancer by administering to the individual a neoantigenic cancer vaccine (NBV) composition in combination with a CD-122-biased cytokine agonist, i.e., RSLAIL-2, optionally further combined with an anti-PD-1 antibody.

### Definitions

In describing and claiming certain features of this disclosure, the following terminology will be used in accordance with the definitions described below unless indicated otherwise.

As used in this specification, the singular forms “a,” “an,” and “the” include plural referents unless the context clearly dictates otherwise.

“Substantially” or “essentially” means nearly totally or completely, for instance, 95% or greater of a given quantity.

Similarly, “about” or “approximately” as used herein means within plus or minus 5% of a given quantity.

It is understood that wherever aspects are described herein with the language “comprising,” otherwise analogous aspects described in terms of “consisting of” and/or “consisting essentially of” are also provided.

In describing and claiming certain features of this disclosure, the following terminology will be used in accordance with the definitions described below unless indicated otherwise.

“PEG” or “polyethylene glycol,” as used herein, is meant to encompass any water-soluble polyethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present invention will comprise one of the two following structures: “—(CH₂CH₂O)ₙ—” or “—(CH₂CH₂CO)ₙ₋₁CH₂CH₂—,” depending upon whether or not the terminal oxygen(s) has been displaced, e.g., during a synthetic transformation. As stated above, for the PEG polymers, the variable (n) ranges from about 3 to 4000, and the terminal groups and architecture of the overall PEG can vary.

“Branched,” in reference to the geometry or overall structure of a polymer, refers to a polymer having two or more polymer “arms” or “chains” extending from a branch point or central structural feature.

A covalent “releasable” linkage, for example, in the context of a polyethylene glycol that is covalently attached to an active moiety such as interleukin-2, is one that releases under physiological conditions by any suitable release mechanism to thereby release or detach a polyethylene glycol polymer from the active moiety.

“Substantially” or “essentially” means nearly totally or completely, for instance, 95% or greater of a given quantity.

Molecular weight in the context of a water-soluble polymer, such as PEG, can be expressed as either a number average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation, or viscometry to determine weight average molecular weight. PEG polymers are typically poly disperse (i.e., number average molecular weight and weight average molecular weight of the polymers are not equal), possessing low polydispersity values of preferably less than about 1.2, more preferably less than about 1.15, still more preferably less than about 1.10, yet still more preferably less than about 1.05, and most preferably less than about 1.03.

The terms “protein”, “polypeptide” and “peptide” are used interchangeably herein and refer to any peptide-linked chain of amino acids, regardless of length co-translational or post-translational modification.

“Pharmaceutically acceptable excipient” or “pharmaceutically acceptable carrier” refers to a component that may be included in the compositions described herein and causes no significant adverse toxicological effects to a subject.

The term “patient,” or “subject” as used herein refers to a living organism suffering from or prone to a condition that can be prevented or treated by administration of a compound or composition or combination as provided herein, such as a cancer, and includes both humans and animals. Subjects include, but are not limited to, mammals (e.g., murines, simians, equines, bovines, porcines, canines, felines, and the like), and preferably are human.

“Administering” refers to the delivery of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

A “cancer” refers a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor.

The term “immunotherapy” refers to the treatment of a subject by a method comprising inducing, enhancing, suppressing, or otherwise modifying an immune response.

A “therapeutically effective amount” or “therapeutically effective dosage” of a therapeutic agent is any amount of the agent, when used alone or in combination with another therapeutic agent, is effective to, for example, protect a subject against the onset of a disease or promote disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “substantially homologous” or “substantially identical” means that a particular subject sequence, for example, a mutant sequence, varies from a reference sequence by one or more substitutions, deletions, or additions, the net effect of which does not result in an adverse functional dissimilarity between the reference and subject sequences. For purposes herein, a sequence having greater than 95 percent homology (identity), equivalent biological activity (although not necessarily equivalent strength of biological activity), and equivalent expression characteristics to a given sequence is considered to be substantially homologous (identical). For purposes of determining homology, truncation of the mature sequence should be disregarded.

With regard to polypeptide and polynucleotide sequence comparisons, the phrase “at least 80% sequence identity” may be used herein. This expression refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide. Preferably, the polypeptide in question and the reference polypeptide exhibit the indicated sequence identity over a continuous stretch of 20, 30, 40, 45, 50, 60, 70, 80, 90, 100 or more amino acids or over the entire length of the reference polypeptide. Preferably, the polynucleotide in question and the reference polynucleotide exhibit the indicated sequence identity over a continuous stretch of 60, 90, 120, 135, 150, 180, 210, 240, 270, 300 or more nucleotides or over the entire length of the reference polypeptide.

### Overview

In an effort to address at least some of the shortcomings associated with current anti-cancer vaccine strategies, such as for example, weak immune responses due to suppression in T-cell growing capacity by a cancerous tumor itself, provided herein is a combination immunotherapy method based upon administration of a neoantigenic vaccine composition comprising a vector encoding multiple cancer neoantigens in tandem, and a CD-122-biased agonist, RSLAIL-2, and optionally, nivolumab.

While cytokines such as IL-2, as well as other adjuvants, have been explored to improve anti-tumor responses to cancer vaccines, further enhancements are needed to provide durable, reproducible and effective vaccine-based cancer therapies. Thus, the present disclosure is based, at least in part, on the discovery of a particularly beneficial therapeutic combination comprising a neoantigen-based cancer vaccine composition and a particular long-acting IL-2Rβ-biased agonist, referred to herein as RSLAIL-2, and optionally, an anti-PD-1 antibody, i.e., nivolumab.

11-2 stimulates immune cell proliferation and activation through a receptor-signaling complex containing alpha (IL2Rα, CD25), beta (IL2Rβ, CD122) and common gamma chain receptors (γ_{c′} CD132). At high doses, IL2 binds to heterodimeric IL2Rβγ receptor leading to desired expansion of tumor killing CD8+ memory effector T (CD8 T) cells. However, IL2 also binds to its heterotrimeric receptor IL2Rαβγ with greater affinity, which expands immunosuppressive CD4+, CD25+ regulatory T cells (Tregs), which may lead to an undesirable effect for cancer immunotherapy. Thus, in an effort to overcome one or more drawbacks associated with IL-2-enhanced anti-cancer vaccination strategies, provided herein is a treatment modality that combines therapeutic cancer vaccination using a genetic vaccine encoding cancer neoantigens with administration of a particular long acting IL-2Rαβ-biased agonist, RSLAIL-2. Without being bound by theory, the Applicants have discovered that by utilizing a long-acting IL-2 compound in which a region that interacts with the IL2Rα subunit responsible for activating immunosuppressive Tregs is masked (i.e., its activity suppressed or dampened), i.e., RSLAIL-2, optionally further combined with an anti-PD-1 antibody, nivolumab, vaccination-induced T-cell responses can be selectively expanded to achieve superior therapeutic efficacy, as will become apparent from the instant disclosure and supporting examples, and in particular, Examples 6, 7 and 8.

### Vaccines

The treatment methods provided herein comprise administering a vaccine, i.e., for stimulating a cancer specific-immune response, e.g., innate and adaptive immune responses, for generating host immunity against a cancer. The compositions and methods provided herein find use in, among other things, both clinical and research applications. Administration of the long acting IL-2Rαβ-biased agonist, RSLAIL-2, in combination with the neoantigen-based vaccine composition described herein, optionally further combined with an anti-PD-1 antibody, preferably nivolumab, is effective, for example, to (i) significantly enhance the T-cell response to vaccine-encoded neoantigens, and (iii) increase immune activity against high, medium and low affinity antigens. That is to say, the combination administration regimens described herein significantly increase the immunity conferred by the NBV composition to a subject.

The supporting examples illustrate, among other things, that when compared to administration of the neoantigen-based vaccine composition alone, the combination is effective to provide an immune response against a larger number of vaccine-encoded neoantigens as well as increased numbers of CD4 and CD8 T cells reactive with the vaccine-encoded neoantigens. Moreover, tumors in mice treated with the combination described herein were highly enriched in T-cells reactive to vaccine-encoded neoantigens.

The neoantigen-based vaccine (NBV) composition encodes multiple cancer neoantigens in tandem. Each neoantigen is a polypeptide fragment derived from a protein mutated in cancer cells. More particularly, the NBV composition comprises a first vector comprising a nucleic acid construct encoding multiple immunogenic polypeptide fragments, each of a protein mutated in cancer cells, wherein each immunogenic polypeptide fragment comprises one or more mutated amino acids flanked by a variable number of wild type amino acids from the original protein, joined head-to-tail to form an immunogenic polypeptide, and the lengths of each of the immunogenic polypeptide fragments can vary.

As used herein, the term “vector” refers to at least one polynucleotide or to a mixture of at least one polynucleotide and at least one protein capable of introducing the polynucleotide comprised therein into a cell. At least one polynucleotide comprised by the vector consists of or comprises at least one nucleic acid construct encoding at least one immunogenic protein. In addition to the polynucleotide consisting of or comprising the nucleic acid construct, additional polynucleotides and/or polypeptides may be introduced into the cell. The addition of additional polynucleotides and/or polypeptides is especially desirable if the additional polynucleotides and/or polypeptides are required to introduce the nucleic acid construct into the cell or if the introduction of additional polynucleotides and/or polypeptides increases the expression of the immunogenic polypeptide encoded by the nucleic acid construct.

The following type of genetic mutations identified at a DNA level can form neoantigens: (i) one or more single amino acid changes caused by a point mutation, and/or (ii) a non-wild type amino acid sequence caused by insertions/deletions resulting in frame shifted peptide, and/or (iii) a non-wild-type amino acid sequence caused by alteration of exon boundaries or by mutations generating intron retention, and/or (iv) a mutated cancer protein generated by a gene fusion event.

For example, the nucleic acid construct of the first vector may encode an immunogenic polypeptide comprising at least 1 immunogenic polypeptide fragment, (ii) preferably at least 4 immunogenic fragments, and (iii) more preferably more than 19 polypeptide fragments, each of a protein mutated in cancer cells, joined head-to-tail.

The term “nucleic acid construct” refers to a polynucleotide that encodes at least one immunogenic polypeptide. Preferably, the polynucleotide additionally comprises elements which direct transcription and translation of the at least one polypeptide encoded by the nucleic acid construct. Such elements include promoter and enhancer elements to direct transcription of mRNA in a cell-free or a cell-based based system, preferably a cell-based system, cap structures etc.

As outlined above, the vector may be a viral vector and, thus, the nucleic acid construct is preferably comprised by a larger polynucleotide which additionally includes nucleic acid sequences which are required for the replication of the viral vector and/or regulatory elements directing expression of the immunogenic polypeptide.

The NBV composition may further comprise a second vector comprising a nucleic acid construct encoding multiple immunogenic polypeptide fragments, each of a protein mutated in cancer cells, wherein each immunogenic polypeptide fragment comprises one or more mutated amino acids flanked by a variable number of wild type amino acids from the original protein, joined head-to-tail to form an immunogenic polypeptide, and at least one epitope of the immunogenic polypeptide encoded by the first vector is immunologically identical to at least one epitope of the immunogenic polypeptide encoded by the nucleic acid construct of the second vector, for use in a prime-boost vaccination regimen.

In some particular embodiments, the vector comprises a nucleic acid construct encoding the polypeptide sequence of SEQ ID NO: 1 or encoding a polypeptide sequence having at least 80% sequence identity to SEQ ID NOT.

In particular embodiments, preferred vectors are adenoviral vectors, in particular adenoviral vectors derived from human or non-human great apes and poxy viral vectors, preferably modified vaccinia virus AnkaraMVA.

The NBV composition may be administered by any suitable administration route, for example, intradermal, intravenous, intramuscular, subcutaneous, intranodel, intralymphatic, intratumoral, and the like. In some embodiments, the NBV composition is administered intramuscularly.

### Long Acting, IL-2Rβ-Biased Agonist, RSLAIL-2

The methods, formulations, kits and the like described herein comprise as the long acting, IL-2Rβ-biased agonist, RSLAIL-2 (encompassing pharmaceutically acceptable salt forms thereof), the preparation of which is described in Example 1. RSLAIL-2 exhibits about a 60-fold decrease in affinity to IL-2Rαβ relative to IL-2, but only about a 5-fold decrease in affinity IL-2Rβ relative to IL-2 (as described in Example 2).

The releasable PEG comprised in RSLAIL-2 is based upon a 2,7,9-substituted fluorene as shown below, with poly(ethylene glycol) chains extending from the 2- and 7-positions on the fluorene ring via amide linkages (fluorene-C(O)—NH—), and having releasable covalent attachment to IL-2 via attachment to a carbamate nitrogen atom attached via a methylene group (—CH₂—) to the 9-position of the fluorene ring. In this regard, RSLAIL-2 is a composition comprising compounds encompassed by the following formula:

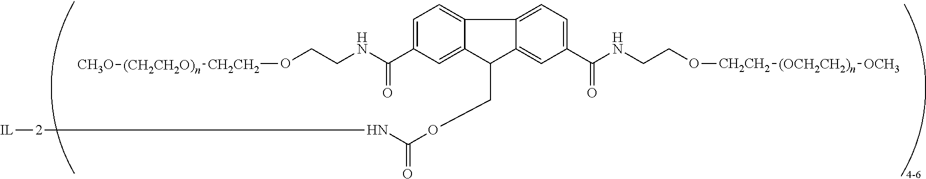

wherein IL-2 is an interleukin-2, and pharmaceutically acceptable salts thereof, where each “n” is an integer from about 3 to about 4000, or more preferably is an integer from about 200-300. In some preferred embodiments, each “n” is approximately the same. That is to say, the weight average molecular weight of each polyethylene glycol “arm” covalently attached to the fluorenyl core is about the same. In some preferred embodiments, the weight average molecular weight of each PEG arm is about 10,000 daltons, such that the weight average molecular weight of the overall branched polymer moiety is about 20,000 daltons. In one or more embodiments, the composition contains no more than 10% (based on a molar amount), and preferably no more than 5% (based on a molar amount), of compounds encompassed by the following formula

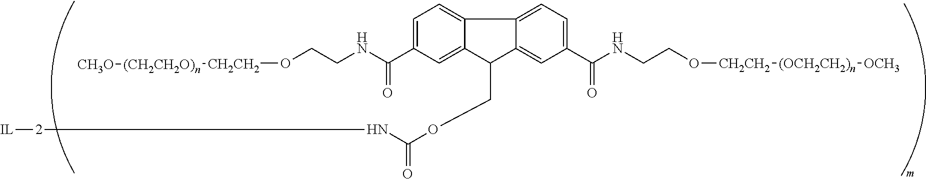

wherein IL-2 is an interleukin-2, and “m” (referring to the number of polyethylene glycol moieties attached to IL-2) is an integer selected from the group consisting of 1, 2, 3, 7 and >7, or pharmaceutically acceptable salts thereof. In some preferred embodiments, RSLAIL-2 possesses on average about six of the branched fluorenyl-based polyethylene glycol moieties attached to IL-2.

To determine average degree of PEGylation for a composition such as described in Formula (I), typically the protein is quantified by a method such as an bicinchoninic acid (BCA) assay or by UV analysis, to determine moles of protein in the sample. The PEG moieties are then released by exposing the sample to conditions in which the PEG moieties are released, and the released PEG is then quantified (e.g., by BCA or UV) and correlated with moles protein to determine average degree of PEGylation.

RSLAIL-2 can be considered to be an inactive prodrug, i.e., it is inactive upon administration, and by virtue of slow release of the polyethylene glycol moieties in vivo, provides active conjugated forms of interleukin-2 that are effective to achieve sustained concentrations at a tumor site.

Additional exemplary compositions of RSLAIL-2 comprise compounds in accordance with the above formulae wherein the overall branched polymer portion of the molecule has a weight average molecular weight in a range of from about 250 Daltons to about 90,000 Daltons. Additional suitable ranges include weight average molecular weights in a range selected from about 1,000 Daltons to about 60,000 Daltons, in a range of from about 5,000 Daltons to about 60,000 Daltons, in a range of about 10,000 Daltons to about 55,000 Daltons, in a range of from about 15,000 Daltons to about 50,000 Daltons, and in a range of from about 20,000 Daltons to about 50,000 Daltons.

Additional illustrative weight-average molecular weights for the polyethylene glycol polymer portion include about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, and about 75,000 Daltons. In some embodiments, the weight-average molecular weight of the branched polyethylene glycol polymer is about 20,000 daltons.

As described above, RSLAIL-2 may be in the form of a pharmaceutically-acceptable salt. Typically, such salts are formed by reaction with a pharmaceutically-acceptable acid or an acid equivalent. The term “pharmaceutically-acceptable salt” in this respect, will generally refer to the relatively non-toxic, inorganic and organic acid addition salts. These salts can be prepared in situ in the administration vehicle or the dosage form manufacturing process, or by separately reacting a long-acting interleukin-2 as described herein with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, oxylate, mesylate, glucoheptonate, lactobionate, and laurylsulphonate salts and the like. (See, for example, Berge et al. (1977) “Pharmaceutical Salts”, J. Pharm. Sci. 66:1-19). Thus, salts as described may be derived from inorganic acids such as hydrochloride, hydrobromic, sulfuric, sulfamic, phosphoric, nitric, and the like; or prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In reference to RSLAIL-2, the term “IL-2” as used herein, refers to a moiety having human IL-2 activity. The term, ‘residue’, in the context of residue of IL-2, when used, means the portion of the IL-2 molecule that remains following covalent attachment to a polymer such as a polyethylene glycol, at one or more covalent attachment sites, as shown in the formula above. It will be understood that when the unmodified IL-2 is attached to a polymer such as polyethylene glycol, the IL-2 is slightly altered due to the presence of one or more covalent bonds associated with linkage to the polymer(s). This slightly altered form of the IL-2 attached to another molecule is sometimes referred to a “residue” of the IL-2.

Proteins having an amino acid sequence corresponding to any one of SEQ ID NOs: 1 through 4 described in International Patent Publication No. WO 2012/065086 are exemplary IL-2 proteins. The term substantially homologous means that a particular subject sequence, for example, a mutant sequence, varies from a reference sequence by one or more substitutions, deletions, or additions, the net effect of which does not result in an adverse functional dissimilarity between the reference and subject sequences. For the purposes herein, sequences having greater than 95 percent homology, equivalent biological activity (although not necessarily equivalent strength of biological activity), and equivalent expression characteristics are considered substantially homologous. For purposes of determining homology, truncation of the mature sequence should be disregarded. The IL-2 may be naturally-occurring or may be recombinantly produced. In addition, the IL-2 can be derived from human sources, animal sources, and plant sources. Most preferably, the IL-2 is aldesleukin.

RSLAIL-2 is generally referred to as long-acting. For the purposes herein, the long acting nature of an IL-2Rβ biased agonist is typically determined using flow cytometry to measure STAT5 phosphorylation in lymphocytes at various time points after administration of the agonist to be evaluated in mice. As a reference, the signal is lost by around 24 hours with IL-2, but is sustained for a period greater than that for a long-acting IL-2R3-biased agonist. As an illustration, the signal is sustained over several days for RSLAIL-2.

Example 2 provides both in-vitro and in-vivo data related to receptor bias for exemplary compositions of RSLAIL-2. As described in Example 2, in a murine melanoma tumor model, the ratio of CD8/regulatory T cells for RSLAIL-2 when compared to IL-2 supports preferential activation of the IL-2 receptor beta over IL2 receptor alpha. RSLAIL-2 is, for example, effective to preferentially activate and expand effector CD8+T- and NK-cells over Tregs.

Moreover, RSLAIL-2 provides increased tumor exposure, and preferably significantly enhanced tumor exposure relative to IL-2, for example, at least a 50-fold increased exposure, or at least a 100-fold increased exposure, or at least a 200-fold increased exposure, or at least a 300-fold increased exposure, or at least a 400-fold increased exposure, or at least a 500-fold increased exposure when normalized for equivalents of IL-2. As an illustration, the antitumor activity of RSLAIL-2 in a mouse melanoma tumor model is described in Example 3. As described therein, RSLAIL-2 was found to provide significantly enhanced tumor exposure, e.g., 500-fold, relative to IL-2 (normalized based upon IL-2 equivalents).

Based upon at least one or more of the features of RSLAIL-2, provided herein are methods effective to selectively expand NBV-composition-induced T-cell responses in cancer patients by administering RSLAIL-2, a long-acting IL-2 compound in which a region that interacts with the IL2Rα subunit responsible for activating immunosuppressive Tregs is masked, to thereby achieve superior therapeutic efficacy.

In accordance with the methods, compositions, and kits described herein, RSLAIL-2 is provided in an IL-2R3-activating amount. One of ordinary skill in the art can determine how much RSLAIL-2 is sufficient to provide clinically relevant agonistic activity at IL-2Rβ. For example, one of ordinary skill in the art can refer to the literature and/or administer a series of increasing amounts of RSLAIL-2 and determine which amount or amounts provide clinically effective agonistic activity of IL-2Rβ. Alternatively, an activating amount of RSLAIL-2 can be determined using the in vivo STAT5 phosphorylation assay where an amount sufficient to induce STAT5 phosphorylation in greater than 10% of NK cells at peak is considered to be an activating amount.

In one or more instances, however, the IL-2Rβ-activating amount of RSLAIL-2 is an amount encompassed by one or more of the following ranges expressed in amount of protein: from about 0.01 to 100 mg/kg; from about 0.01 mg/kg to about 75 mg/kg; from about 0.02 mg/kg to about 60 mg/kg; from about 0.03 mg/kg to about 50 mg/kg; from about 0.05 mg/kg to about 40 mg/kg; from about 0.05 mg/kg to about 30 mg/kg; from about 0.05 mg/kg to about 25 mg/kg; from about 0.05 mg/kg to about 15 mg/kg; from about 0.05 mg/kg to about 10 mg/kg; from about 0.05 mg/kg to about 5 mg/kg; from about 0.05 mg/kg to about 1 mg/kg. In some embodiments, RSLAIL-2 is administered at a dose that is less than or equal to 0.7 mg/kg. Particular illustrative dosing ranges include for example, from about 0.1 mg/kg to about 10 mg/kg, or from about 0.2 mg/kg to about 7 mg/kg or from about 0.2 mg/kg to less than about 0.7 mg/kg.

For confirmation, with respect to RSLAIL-2, the amount and extent of the activation can vary widely and still be effective when coupled with administration of a NBV composition, optionally with an anti-PD-1 antibody, i.e., nivolumab. That is to say, an amount of RSLAIL-2 that exhibits only minimal agonist activity at IL-2Rβ for a sufficiently extended period of time can still be a long-acting, IL-2Rβ-biased agonist so long as when administered with a NBV composition as described herein, optionally with nivolumab, the methods, compositions, and kits described herein enable a clinically meaningful response. In some instances, due to (for example) synergistic interactions and responses, only minimal agonist activity of IL-2Rβ may be required when accompanied by vaccination with the neoantigen-based vaccine composition described herein.

In accordance with the method described herein, a PD-1 pathway-inhibiting amount of an anti-PD-1 antibody, i.e., nivolumab may be administered. One of ordinary skill in the art can determine how much anti-PD-1 antibody, such as nivolumab, is sufficient to provide clinically relevant inhibition of the PD-1 pathway. For example, one of ordinary skill in the art can refer to the literature and/or administer a series of increasing amounts of nivolumab and determine which amount or amounts provide clinically relevant inhibition of the PD-1 pathway.

In one or more instances, however, the PD-1 pathway-inhibiting amounts of the anti-PD-1 antibody, i.e., nivolumab, are encompassed by one or more of the following ranges: from about 1 mg/kg to about 1000 mg/kg; from about 2 mg/kg to about 900 mg/kg; from about 3 mg/kg to about 800 mg/kg; from about 4 mg/kg to about 700 mg/kg; from about 5 mg/kg to about 600 mg/kg; from about 6 mg/kg to about 550 mg/kg; from about 7 mg/kg to about 500 mg/kg; from about 8 mg/kg to about 450 mg/kg; from about 9 mg/kg to about 400 mg/kg; from about 5 mg/kg to about 200 mg/kg; from about 2 mg/kg to about 150 mg/kg; from about 5 mg/kg to about 100 mg/kg; from about 10 mg/kg to about 100 mg/kg; and from about 10 mg/kg to about 60 mg/kg.

For confirmation, as used herein with regard to PD-1 pathway-inhibiting amounts of an anti-PD-1 antibody such as nivolumab, the amount and extent of the inhibition can vary widely and can still be effective, even when combined with administration of RSLAIL-2 and the NBV composition. For example, an amount of an anti-PD-1 antibody, i.e., nivolumab, that only minimally inhibits the PD-1 pathway can still be an inhibiting amount as used herein so long as the method results in a clinically meaningful response. The actual dose to be administered, for each of the immunotherapeutic components, will vary depend upon the age, weight, and general condition of the subject as well as the severity of the condition being treated, the judgment of the health care professional, and conjugate being administered.

In certain embodiments, the subject has received one, two, three, four, five or more prior cancer treatments. In other embodiments, the subject is treatment-naïve. In some embodiments, the subject has progressed on other cancer treatments. In certain embodiments, the prior cancer treatment comprised an immunotherapy. In other embodiments, the prior cancer treatment comprised a chemotherapy. In some embodiments, the tumor has reoccurred. In some embodiments, the tumor is metastatic. In other embodiments, the tumor is not metastatic.

In some embodiments, the subject has received a prior therapy to treat the tumor and the tumor is relapsed or refractory. In some embodiments, the subject has received a prior immuno-oncology therapy to treat the tumor and the tumor is relapsed or refractory. In some embodiments, the subject has received more than one prior therapy to treat the tumor and the subject is relapsed or refractory.

In some treatment modalities, an NBV composition is first administered for priming an immune response in the subject, followed by a subsequent administration of the same NBV composition or a neoantigen vaccine composition directed against the same disease for boosting the immune response in the subject. The boost may be administered within 1 week, 2 weeks, 3 weeks or 4 weeks or more following the first administering (or priming).

The treatment methods described herein can continue for as long as the clinician overseeing the patient's care deems the treatment method to be effective. Non-limiting parameters that indicate the treatment method is effective include any one or more of the following: tumor shrinkage (in terms of weight and/or volume); a decrease in the number of individual tumor colonies; tumor elimination; and progression-free survival. Change in tumor size may be determined by any suitable method such as imaging. Various diagnostic imaging modalities can be employed, such as computed tomography (CT scan), dual energy CDT, positron emission tomography and MRI.

With regard to the frequency and schedule of administering the NBV composition and RSLAIL-2, optionally with an anti-PD-1 antibody, nivolumab, one of ordinary skill in the art will be able to determine an appropriate frequency for each of the components of the combination. For example, in a treatment cycle, a clinician can decide to administer the NBVV composition, either as a single dose or in a series of doses, e.g., over the course of several days or weeks). RSLAIL-2 is administered, either concurrently with the NBV composition or prior to vaccination, or following administration of the genetic cancer vaccine. For example, in some treatment modalities, RSLAIL-2 is administered within 7 days of NBV composition administration (e.g., on any one of days 1, 2, 3, 4, 5, 6, or 7). In some instances, RSLAIL-2 is administered within 4 days of vaccination, e.g., on any one of days 1, 2, 3, or 4. In some other embodiments, the NBV composition is administered on day 1 of treatment and RSLAIL-2 is administered on a day greater than 5 days following vaccination (e.g., on day 6, 7, 8, 9, 10, 11, 12, 13, 14, or 15 of treatment), particularly in an early therapeutic setting prior to growth of established tumors.

Based upon the long acting nature of RSLAIL-2, such composition may be administered relatively infrequently (e.g., once every three weeks, once every two weeks, once every 8-10 days, once every week, etc.).

Exemplary lengths of time associated with the course of therapy include about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years.

The treatment methods described herein are typically continued for as long as the clinician overseeing the patient's care deems the treatment method to be effective, i.e., that the patient is responding to treatment. Non-limiting parameters that indicate the treatment method is effective may include one or more of the following: tumor shrinkage (in terms of weight and/or volume and/or visual appearance); a decrease in the number of individual tumor colonies; tumor elimination; progression-free survival; appropriate response by a suitable tumor marker (if applicable), increased number of NK (natural killer) cells, increased number of T cells, increased number of memory T cells, increased number of central memory T cells, reduced numbers of regulatory T cells such as CD4+ Tregs, CD25+ Tregs, and FoxP3+ Tregs.

The methods provided herein are useful for (among other things) treating a patient suffering from cancer. For example, patients may be responsive to the NBV composition alone, as well as the combination with RSLAIL-2, optionally with an anti-PD-1 antibody, i.e., nivolumab, but are more responsive to the combination. By way of further example, patients may be non-responsive or only marginally responsive to either the NBV composition or to RSLAIL-2 or to the anti-PD-1 antibody, i.e., nivolumab, but are more responsive to the combination. By way of still further example, patients may be non-responsive to the NBV composition or to RSLAIL-2 or to the anti-PD-1 antibody (i.e., nivolumab) alone, but are responsive to the combination.

Administration, e.g., of the NBV composition and/or RSLAIL-2 and/or the anti-PD-1 antibody is typically via injection. Other modes of administration are also contemplated, such as pulmonary, nasal, buccal, rectal, sublingual and transdermal. As used herein, the term “parenteral” includes subcutaneous, intravenous, intra-arterial, intratumoral, intralymphatic, intraperitoneal, intracardiac, intrathecal, and intramuscular injection, as well as infusion injections. As described previously, the NBV composition and RSLAIL-2 and optionally the anti-PD-1 antibody, i.e., nivolumab can be administered separately. Alternatively, if administration of the NBV composition and RSLAIL-2 and optionally, an anti-PD-1 antibody such as nivolumab, is desired to be simultaneous, either as an initial dose or throughout the course of treatment or at various stages of the dosing regimen—and each of the components are compatible together and in a given formulation—then the simultaneous administration can be achieved via administration of single dosage form/formulation (e.g., intravenous administration of an intravenous formulation that contains the two or three immunological components). One of ordinary skill in the art can determine through routing testing whether two such components are compatible together and in a given formulation. For example, administration to a patient can be achieved through injection of a composition comprising RSLAIL-2 and a diluent. In addition, administration to a patient can be achieved through injection of the NBV composition and a diluent. Further, administration can be achieved through injection of a composition comprising RSLAIL-2, a NBV composition and optionally an anti-PD-1 antibody such as nivolumab, and a diluent. With respect to possible diluents, the diluent can be selected from the group consisting of bacteriostatic water for injection, dextrose 5% in water, phosphate-buffered saline, Ringer's solution, lactated Ringer's solution, saline, sterile water, deionized water, and combinations thereof. One of ordinary skill in the art can determine through routing testing whether two given pharmacological components are compatible together in a given formulation.

The therapeutic combination described herein, i.e., the NBV composition, RSLAIL-2 and optionally an anti-PD-1 antibody, i.e., nivolumab, may be provided in the form of a kit. As described above, the components may be comprised in a single composition, optionally accompanied by one or more pharmaceutically acceptable excipients, or may be provided in separate containers, where the kit typically includes instructions for use. Suitable pharmaceutically acceptable excipients include those described, for example, in the Handbook of Pharmaceutical Excipients, 7^{th} ed., Rowe, R. C., Ed., Pharmaceutical Press, 2012. The kit components, e.g., compositions comprising the NBV composition, RSLAIL-2 and optionally, an anti-PD-1 antibody, i.e., nivolumab, may be in either liquid or in solid form. In certain preferred embodiments, each immunological component is in solid form. Preferred solid forms are those that are solid dry forms, e.g., containing less than 5 percent by weight water, or preferably less than 2 percent by weight water. The solid forms are generally suitable for reconstitution in an aqueous diluent.

The presently described methods, kits and related compositions can be used to treat a patient suffering from any condition that can be remedied or prevented by the methods provided herein, such as cancer. Exemplary conditions are cancers, such as, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, brain cancer, breast cancer, ovarian cancer, prostate cancer, squamous cell cancer, basal cell cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, embryonal cancer, Wilms' tumor, cervical cancer, Hodgkin lymphoma, non-Hodgkin lymphoma, testicular cancer, lung cancer, small cell lung cancer, brain cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, melanoma, multiple myeloma, neuroblastoma, retinoblastoma and leukemias. In some particular embodiments, the cancer to be treated is a solid cancer, such as for example, breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease and adrenocortical cancer.

The present methods, kits and compositions are useful for enhancing the therapeutic effectiveness of a NBV composition, for example, by improving the subject's response to the neoantigen-based vaccine composition. An enhanced response may be evaluated at any suitable time point during treatment, after a single round of treatment, after 2-3 cycles of treatment, etc., and by any of a number of suitable methods, including shrinkage of a tumor (partial response), i.e., an evaluation of tumor size or volume, disappearance of a tumor, a reduction in disease progression (cancer has not progressed), and analysis of one or more tumor test markers if appropriate. The methods, kits, compositions and the like provided herein are also useful for reducing tumor growth or size (or volume) in a subject undergoing treatment. Treatment by administering a therapeutically effective amount of cancer vaccine and a long-acting IL-2R3-biased agonist such as provided herein to a subject having established tumors is effective, in one or more embodiments, to reduce tumor growth or size in the subject. For example, in some embodiments, one or more cycles of treatment is effective to reduce tumor size by about 25%, or by about 30%, or by about 40%, or by about 50%, or even by about 60%, or by about 70% or more when compared to the size of the tumor prior to treatment.

In yet some further embodiments, the methods, kits, compositions and the like provided herein are effective to inhibit accumulation of regulatory T cells (Tregs) in a subject undergoing treatment for cancer. In some embodiments, the method is effective, for example, when evaluated in a cancer mouse model of the corresponding cancer, to inhibit accumulation of regulatory T cells selected from the group consisting of CD4+ Tregs, CD25+ Tregs, and FoxP3+ Tregs in the tumor (i.e., any one or more of the foregoing cell types) by an amount that is enhanced over that observed upon administration of any one of the immunological components, i.e., the NBV composition, RSLAIL-2, or an anti-PD-1 antibody, i.e., nivolumab, singly. For example, the subject Tregs (measured either singly or as any one of the possible combinations of Tregs) may be inhibited by 1.5-fold or more, or 2-fold or more, or 3-fold or more, or even 4-fold or more, when compared to treatment by any one single immunotherapeutic agent. The treatment may, in some embodiments, be effective to inhibit accumulation of regulatory T cells (Tregs) in a subject by at least 2-fold or more, or 3-fold or more, or even 4-fold or more, or 5-fold or more, or 6-fold or more when compared to an untreated subject.

In yet some further embodiments, the methods, kits, composition and the like provided herein are effective to stimulate T cell and/or NK cell activity and/or proliferation in a subject. In some embodiments, the method is effective, for example, when evaluated in a cancer mouse model of the corresponding cancer, for increasing the number of CD8+ Tcells and/or CD4 T cells in the subject when compared to vaccination with a NBV composition alone. For example, the subject's CD8+ T cells may be increased by 1.5-fold or more, or 2-fold or more, or 3-fold or more, or even 4-fold or more, when compared to treatment with the NBV composition alone or with unmodified IL-2 as an adjuvant. The treatment may, in some embodiments, be effective to increase the subject's CD8+ Tcells or CD4 T cells against the neoantigens by at least 2-fold or more, or 3-fold or more, or even 4-fold or more, or 5-fold or more, or 6-fold or more when compared to an untreated subject.

In turning to the Examples, at least Examples 6-8 provide further indication of the synergy arising from the administration of a NBV composition accompanied by administration of RSLAIL-2, and as shown in Example 8, an illustrative anti-PD-1 antibody (anti-mouse PD-1, clone RMP1-14). In an illustrative mouse tumor model, immunotherapeutic treatment with a combination of a NBV composition, RSLAIL-2 and an anti-PD-1 antibody notably resulted in complete regression of established tumors in all treated animals, demonstrating the unexpected superiority of this combination modality.

All articles, books, patents, patent publications and other publications referenced herein are incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

### EXAMPLES

It is to be understood that the foregoing description as well as the examples that follow are intended to illustrate and not limit the scope of the invention(s) provided herein. Other aspects, advantages and modifications within the scope of the invention will be apparent to those skilled in the art to which the invention pertains.

### Materials and Methods

Recombinant human IL-2 having an amino acid sequence identical to that of aldesleukin was cloned and expressed and used to prepare the exemplary long acting IL-2Rαβ-biased agonist referred to herein as RSLAIL-2.

RSLAIL-2 refers to a composition obtainable upon following the procedures of Example 1 in PCT Int. Pat. Appl. Pub. No. WO 2015/125159, and generically refers to a composition comprising multiPEGylated forms of interleukin-2, wherein attachment of the branched PEG reagent used to form the conjugates is releasable following administration to a subject. The preparation of RSLAIL-2 is described below.

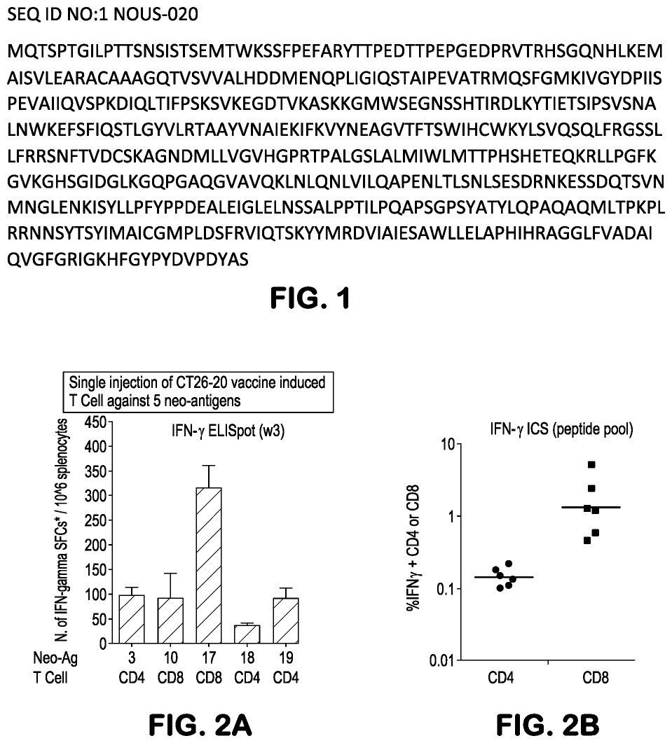

NOUS-020 Neoantigenic Vaccine: NOUS-020 constructs contain 20 non-synonymous single nucleotide variants (SNV) from the CT-26 murine tumor cell line. NOUS-020 vaccine is based on Great Apes derived Adenovirus and MVA vaccines encoding for 20 neoantigens from CT26 murine tumor cell line. The NOUS-020 insert sequence is shown in FIG. 1. For each mutation, the amino acid change is embedded in the wild type protein sequence and flanked both upstream and downstream with 12 amino acids for a total length of 25 amino acids for the neoantigen. The sequences of the protein fragments from different neoantigens are joined head to tail to form the artificial antigen fused downstream with an HA peptide sequence for the purpose of monitoring expression of the recombinant artificial protein.

### Example 1

### Preparation of “RSLAIL-2”, (2,7-(Bis-MethoxyPEG_{10KD}-Carboxyamide)(9H-Fluorene-9-yl)Methyl N-Carbamate)₄₋₆Interleukin-2

Purified rIL-2 (106.4 mL) at 1.44 mg/ml was charged into a first vessel followed by the addition of 53.6 mL of formulation buffer (10 mM sodium acetate, pH 4.5, 5% trehalose). The pH was measured at 4.62 the temperature was measured at 21.2° C. The PEG reagent, C2-PEG2-FMOC-NHS-20K (available as described in WO 2006/138572) (13.1 g), was charged into a second vessel followed by the addition of 73.3 mL of 2 mM HCl. The resulting solution was swirled by hand for 25 minutes. Sodium borate (0.5 M, pH 9.8) was added to the first vessel to raise the pH to about 9.1 and then the contents of the second vessel containing the PEG reagent was added to the first vessel over a period of from one to two minutes. A rinse step was then performed by charging 8.1 mL of 2 mM HCl into the second vessel and adding the contents to the first vessel. For the conjugation reaction, the final rIL-2 concentration was 0.6 mg/mL, the sodium borate concentration was 120 mM, the pH was 9.1+/−0.2, the temperature was 20-22° C., and the molar ratio of PEG reagent to rIL-2, after adjustment for activity of the reagent (substitution level) was 35:1. The conjugation reaction was allowed to proceed for thirty minutes and quenched by acidification by addition of 75 mL of 2N acetic acid (to bring the pH down to approximately to 4).

The product was purified by ion exchange chromatography as previously described to provide a composition of primarily 4-mers, 5-mers and 6-mers (referring to the number of PEG reagents releasably covalently attached to r-IL-2 (wherein 8-mers and conjugates having a higher degrees of PEGylation were removed during a washing step associated with chromatography). This composition is referred to herein as “RSLAIL-2.”

### Example 2

### Receptor-Bias of RSLAIL-2 and Related Immunotherapeutic Properties

Binding Affinity to IL-2 Receptors and Receptor Bias Related to Immunostimulatory Profile: The affinity of RSLAIL-2 to IL-2Rα and IL-2Rβ was measured directly by surface plasmon resonance (Biacore T-100) and compared to that of clinically available IL-2 (aldesleukin). Antihuman antibody (Invitrogen) was coupled to the surface of a CM-5 sensor chip using EDC/NHS chemistry. Then either human IL-2Rα-Fc or IL-2Rβ-Fc fusion protein was used as the captured ligand over this surface. Serial dilutions of RSLAIL-2 and its active IL-2 conjugate metabolites (1-PEG- and 2-PEG-IL-2) were carried out in acetate buffer pH 4.5, starting at 5 mM. These dilutions were allowed to bind to the ligands for 5 minutes, and the response units (RU) bound was plotted against concentration to determine EC50 values. The affinities of each isoform to each IL-2 receptor subtype were calculated as fold change relative to those of IL-2.

The in vitro binding and activation profiles of RSLAIL-2 suggested that PEGylation interferes with the interaction between IL2 and IL2Rα relative to aldesleukin; an investigation was carried out to determine whether these effects bias the profile of immune cell subtypes in vivo. The number of CD8 T and Treg cells in a tumor following administration of either RSLAIL-2 or IL2 provides a measure of whether pleiotropic effects of IL2 have been shifted due to conjugation of IL2 to poly(ethylene glycol) (as in RSLAIL-2) at the IL2/IL2Rα interface. To address the question, mice bearing subcutaneous B16F10 mouse melanoma tumors were treated with a single dose of RSLAIL-2 or 5 doses of aldesleukin, and immune cells in the tumor microenvironment were quantified by flow cytometry.

In tumors of aldesleukin-treated mice, total and memory CD8 cells were increased as a percentage of tumor-infiltrating lymphocytes; however, these effects were transient, reaching significance relative to vehicle on day 5. In contrast, significant (P<0.05) and sustained total and memory CD8 T-cell stimulation was achieved following a single RSLAIL-2 administration, with superior percentages of memory CD8 (day 7) and total CD8 (days 7 and 10) relative to aldesleukin. Both RSLAIL-2 and aldesleukin treatment resulted in increased activated natural killer (NK) cells 5 and 7 days after treatment initiation, though this effect was diminished by day 10. CD4 cell percentages of tumor-infiltrating lymphocytes were diminished following RSLAIL-2 treatment relative to vehicle on day 5. On day 10, RSLAIL-2 resulted in fewer CD4 cell percentages compared with vehicle and aldesleukin. The CD4 cell population was further analyzed for the FoxP3⁺ subset, which defines the Treg population. RSLAIL-2 administration reduced percentage of Tregs at every time point, consistent with reduced access to the IL2Rα subunit arising from the PEG chains. In contrast, Treg reduction with aldesleukin was modest achieving significance on day 5. The increase of CD8 T cells and reduction of Tregs led to a marked elevation of the CD8/Treg ratio in the tumor by day 7. The ratio of CD8/Treg for RSLAIL-2, aldesleukin, and vehicle was 449, 18, and 4, respectively, supporting preferential activation of the IL2 receptor beta over IL2 receptor alpha for RSLAIL-2.

Immunohistochemical staining was performed and confirmed that CD8 T cells were not only increased in number but were interspersed with tumor cells. These results indicate RSLAIL-2 is effective to induce a more robust in vivo memory effector CD8 T-cell response than seen with unmodified IL-2 (aldesleukin), without a commensurate stimulation of Tregs in tumor, consistent with an in vitro IL2Rβ-biased binding profile. Thus, RSLAIL-2 is effective to preferentially activate and expand effector CD8+T- and NK-cells over Tregs.

### Example 3

### Tumor Exposure of RSLAIL-2

The objective of this study was to evaluate the antitumor activity of RSLAIL-2 as a single agent in C57BL/6 mice implanted with B16F10 melanoma cells when compared to aldesleukin.

C57BL/6 mice were implanted subcutaneously into the right flank with B16F10 melanoma cells (1×10⁶ per animal). Seven days after implantation, when tumors measured 200 mm³, animals were administered RSLAIL-2 (2 mg/kg×1) or aldesleukin (3 mg/kg daily×5). Tumors were harvested (n=4 per observation time), homogenized in ice-cold PBS containing protease inhibitor (Roche) and 0.25% acetic acid, and centrifuged to obtain supernatant. To quantify RSLAIL-2 levels in tumor tissue, PEG was released from IL2 by incubating the supernatant in a pH 9 buffer at 37° C. overnight. IL2 was measured by sandwich ELISA specific for human IL2. To calculate AUC, data were fit with Phoenix WinNonLin using a noncompartmental model. AUC after aldesleukin was estimated on the basis of day 1 AUC multiplied by 5.

Tumor aldesleukin levels rapidly reached Cₘₐₓ and then rapidly declined, leading to <4 ng/g concentrations 24 hours after each dose and a daily AUC of 0.09±0.02 μg/hour/g. In contrast, RSLAIL-2 was detectable in tumors for up to 8 days after a single dose achieving an AUC of 30±6.9 μg/hour/g. On the basis of AUC, a single dose of RSLAIL-2 led to a 67-fold higher exposure compared with 5 daily doses of aldesleukin, even though 7.5 fewer IL2 equivalents were dosed using RSLAIL-2 (3 mg/kg daily×5=15 mg/kg vs. 2 mg/kg). Thus, normalizing exposure on the basis of IL2 equivalents, RSLAIL-2 achieved a 500-fold increased exposure relative to aldesleukin. The active conjugated IL-2 form of RSLAIL-2 (2-PEG-IL2 and 1-PEG-IL2 together) was also quantified and remained detectable in tumor for up to 5 days yielding an AUC of 23±4.4 μg/g. Hence, exposure to active conjugated IL-2 was 50-times higher compared with aldesleukin, translating to 380 times increased exposure relative to an equivalent dose of aldesleukin. The tumor exposure of RSLAIL-2 thus allowed dosing once every 9 days in mice compared with twice daily for two 5-day cycles for aldesleukin.

### Example 4

### Immunogenicity of NOUS-020 GAd Vaccine

The immunogenicity of the NOUS-020 GAd vaccine was evaluated in BALB/c inbred mice after single intramuscular immunization at dose of 5×10⁸ viral particles (vp). Splenocytes were collected three weeks post-immunization and tested by IFN-γ ELISpot by stimulating cells in the presence of synthetic peptides corresponding to each neoantigen vaccine encoded. Negative-control cultures included cells stimulated with culture medium alone in presence of peptide diluent dimethyl sulfoxide (DMSO). Immune responses (number of T cells producing IFN-γ per million splenocytes) are shown in the related figures. Responses were considered positive if the mean of antigen wells was greater than 15 SFC/10⁶ PBMC and exceeded by 3-fold the background value of DMSO wells. Quality of T cell responses (CD4 and CD8) was measured by Intracellular IFN-γ cytokine staining with a pool of 5 neo-antigens that were immunogenic in the ELISpot assay. Immune responses (number of T cells producing IFN-γ per million splenocytes) are shown in FIGS. 2A and 2B. As can be seen, NOUS-020 GAd vaccine induces CD4 and CD8 T cells.

### Example 5

### Immunogenicity of NOUS-020 GAd-MVA Vaccine

The immunogenicity of the NOUS-020 GAd-MVA vaccine was evaluated in prime-boost studies. BALB/c inbred mice were primed with GAd (dose of 5×10⁸ viral particles) and then boosted with MV A (10⁷ pfu) at week 4. Vaccine-induced responses were measured one week post boost by IFN-γ ELISpot stimulating spleen cells with a pool of 20 vaccine encoded neoantigens. Negative-control cultures included cells stimulated with culture medium alone in the presence of the peptide diluent, dimethyl sulfoxide (DMSO).

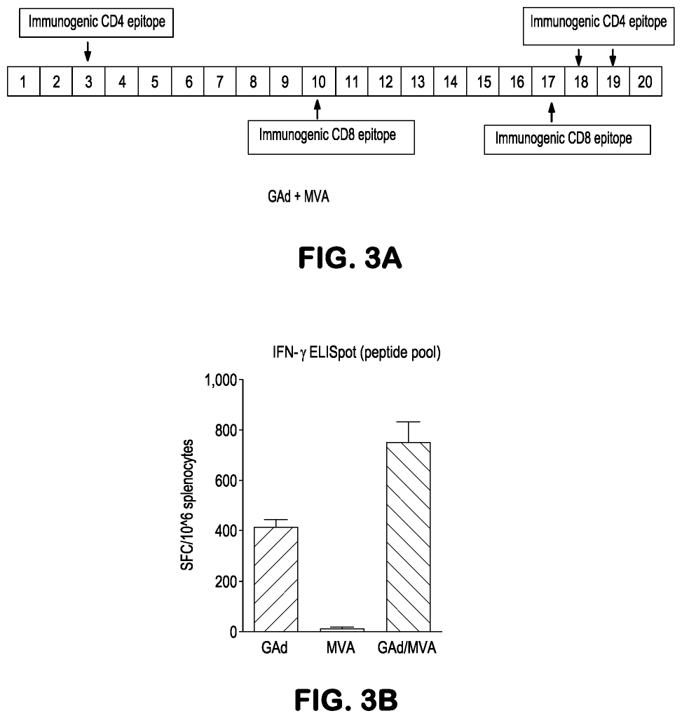

Immune responses (number of T cells producing IFN-γ per million splenocytes) are shown in FIGS. 3B and 3C. Responses were considered positive if the mean of antigen wells was greater than 15 SFC/10⁶ PBMC and exceeded by 3 fold the background value of DMSO wells. Quality of T cell responses (CD4 and CD8) was measured by Intracellular IFN-γ cytokine staining with a pool of 5 neo-antigens resulted immunogenic for ELISpot assay. The quality of T cell responses (CD4 and CD8) was measured by Intracellular IFN-γ cytokine staining with a pool of top 5 neo-antigens resulted immunogenic for ELISpot assay. As shown, the neoantigenic vaccine induced a response for CD4 and CD8 T-cells. FIG. 3A provides a schematic of constructs showing the neoantigens that induce a CD8 and CD4 response. FIG. 3B provides an analysis of T cell responses measured post GAd/MVA immunization in naive mice by IFN-γ ELISpot on pool of 20 vaccine encoded neo-antigens.

### Example 6

### Combination Treatment with NOUS-020 GAd-CT26 Neoantigenic Vaccine and RSLAIL-2 in a Murine CT26 Tumor Model (Early Therapeutic Setting)

The therapeutic efficacy of concomitant administration (NOUS-020 GAd vaccine and RSLAIL-2 at Day 0) and subsequent administration (GAd Day 0, and RSLAIL-2, Day7) of NOUS-020 vaccine and RSLAIL-2 was evaluated on CT26 tumor growth in BALB/c mice.

BALB/c mice were injected with CT26 colon carcinoma cells at Day −3. Three days later (Day 0), mice were treated with (i) NOUS-020 GAd vaccine alone (intramuscularly, at a dose of 5×10⁸ viral particles), (ii) RSLAIL-2 alone (intravenously, 0.8 mg/kg, q9x3), or a combination of NOUS-020 GAd vaccine and RSLAIL-2 administered either (iii) concomitantly at Day 0 or (iv) with a sequential administration regimen with NOUS-020 GAd vaccine administered at Day 0 and RSLAIL-2 administered at Day 7.

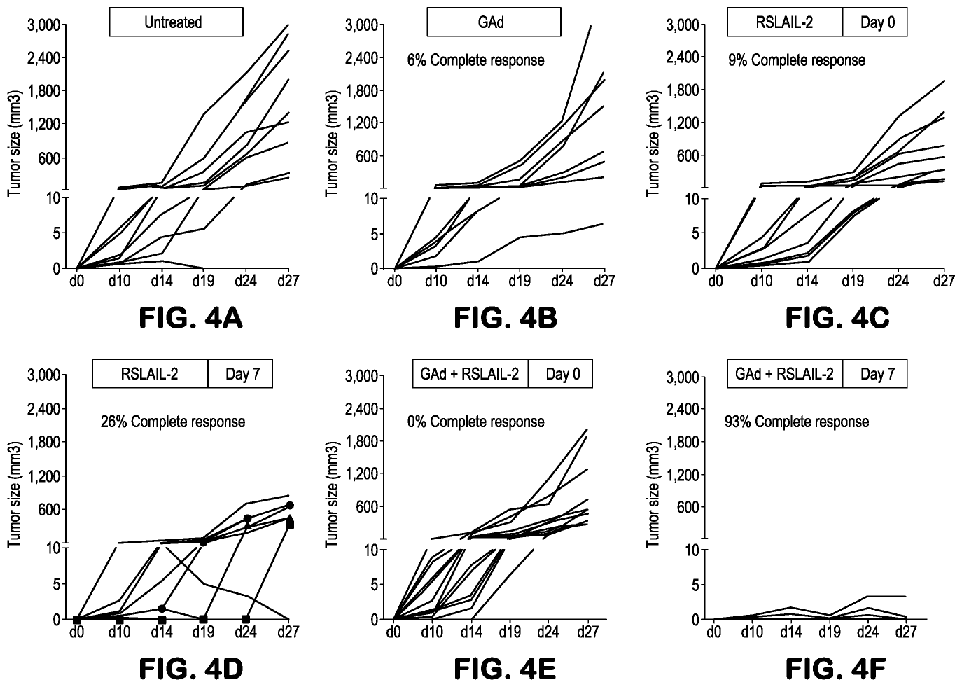

Tumor volumes were recorded over time for each treatment group. Results are shown in FIGS. 4A-4F. FIG. 4A provides results for the control group (untreated); FIG. 4B demonstrates volume of CT26 tumors in mice treated with GAd vaccine alone; FIGS. 4C and 4D demonstrate volume of CT26 tumors in mice treated with RSLAIL-2 (administered at either day 0 or 7, respectively) and concomitant at Day 0 (FIG. 4E) or sequential administration (FIG. 4F) of RSLAIL-2 and GAd, respectively.

As can be seen from the figures, administration of RSLAIL-2 on day 7 notably improved the efficacy of the NOUS-020 GAd vaccine in an early therapeutic setting (i.e., prior to growth of a sizable tumorous mass).

To further explore various treatment regimens for administering NOUS-020 GAd vaccine and RSLAIL-2 in combination, different treatment intervals were explored.

BALB/c mice were challenged with CT26 tumor cells and 3 days post challenge (day 0) they received NOUS-020 GAd vaccine (day 0, 5×10⁸ viral particles) or a combination of NOUS-020 GAd administered at day 0 and RSLAIL-2 administered either at day 3, 5, or 7. Tumor growth was monitored over time in the various treatment groups. Table 1 below shows the percentage of tumor-free mice at the end of the study for each treatment modality.

TABLE 1
Dosing Regimen Versus Complete Response

| | Percent Tumor Free Mice |
|---|---|
| Regimen | (Percent Complete Response) |
| NOUS-020 GAd | 6% |
| NOUS-020 GAd + RSAIL-2 (Day 3) | 28% |
| NOUS-020 GAd + RSAIL-2 (Day 5) | 70% |
| NOUS-020 GAd + RSAIL-2 (Day 7) | 80% |

Based upon the data in Table 1, it appears that the interval between GAd vaccination and single dose administration of RSAIL-2 affects synergic activity. Based on the data above, it appears that RSAIL-2 is preferably first administered at more than 5 days following vaccination, for example, at 6 days, or at 7 days, or at 8 days, or at 9 days or at 10 days or more following vaccination.

### Example 7

### Combination Treatment of Established Tumors with NOUS-020 GAd-MVA CT26 Neoantigenic Vaccine and RSLAIL-2 in a Murine CT26 Tumor Model

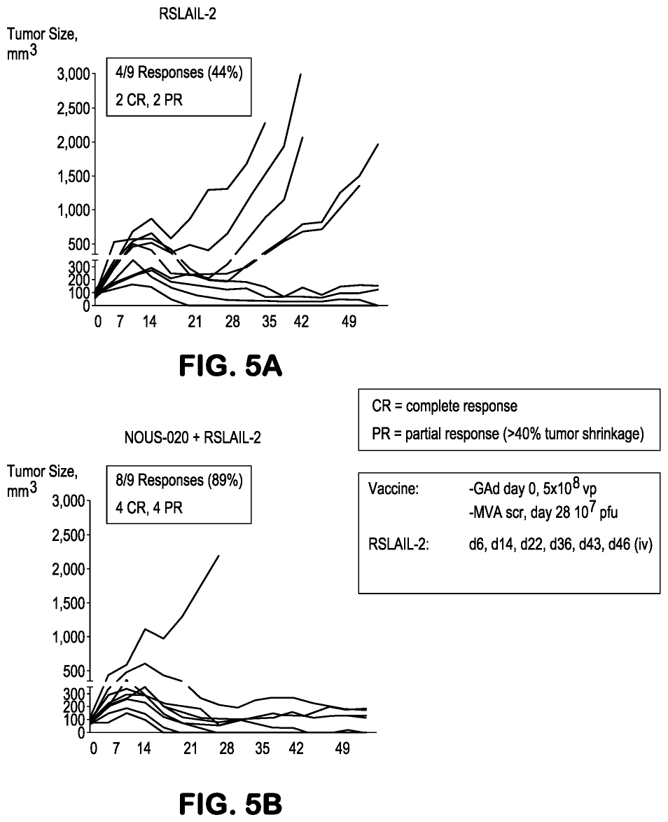

BALB/c mice were challenged with CT26 cells. After a week, mice having a tumor mass of 100 mm³ were randomized into 2 groups (day 0), one group receiving RSLAIL-2 alone, the second group receiving a combination of NOUS-020 and RSLAIL-2, respectively, administered at day 0 (5×10⁸ viral particles) and day 6 (intravenous, 0.8 mg/kg). Administration of RSAIL-2 was repeated at day 14, day 22, day 36, day 43, and day 46. Boost with MVA for the group receiving the combination treatment was performed at day 28, with intramuscular injection of MVA at a dose of 10⁷ pfu. Tumor volumes were monitored over time. Results are shown in FIGS. 5A (RSAIL-2 only) and 5B (NOUS-020 and RSAIL-2). The group administered RSLAIL-2 alone had a 44% response rate with 2 complete responders and 2 partial responders (partial response=greater than 40% tumor shrinkage, but not complete disappearance of tumor). In contrast, the group administered a combination of NOUS-020 and RSLAIL-2 as described above had an 89% response to treatment, with 4 complete responders and 4 partial responders.

These results demonstrate that combined therapy with NOUS-020 vaccine and RSLAIL-2 is effective in treating mice with established tumors.

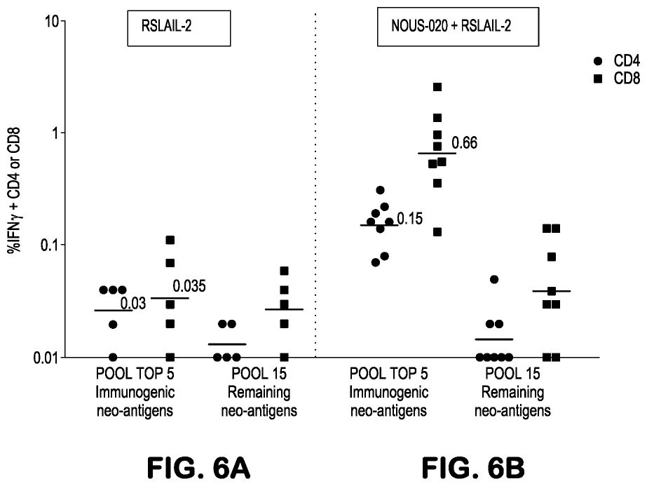

Responder mice from the combination group NOUS-020 and RSLAIL-2 or RSLAIL-2 only were sacrificed at day 54. An assessment of antigen-specific T cell responses was performed by intracellular IFN-γ staining on spleen cells stimulated in the presence of two separate peptide pools: a pool of top 5 immunogenic neo-peptides and a second pool containing the remaining 15 vaccine encoded neo-peptides. Results are shown in FIGS. 6A (RSLAIL-2 only) and 6B (NOUS-020 and RSLAIL-2).

### Example 8

### Combination Treatment of Established Tumors with NOUS-020 GAd-MVA CT26 Neoantigenic Vaccine, RSLAIL-2, and Anti-PD-1 in a Murine CT26 Tumor Model

BALB/c mice were challenged with CT26 cells. After a week, mice having a tumor mass of about 100 mm³ were randomized into 5 groups as follows: (i) anti-PD-1 (clone RMP1-14), (ii) RSLAIL-2, (iii) anti-PD-1 (clone RMP1-14) and RSLAIL-2, (iv) NOUS-020 vaccine (GAd, MV A)+RSLAIL-2 and (v) anti-PD-1 (clone RMP1-14), NOUS-020 vaccine (GAd, MV A) and RSLAIL-2.

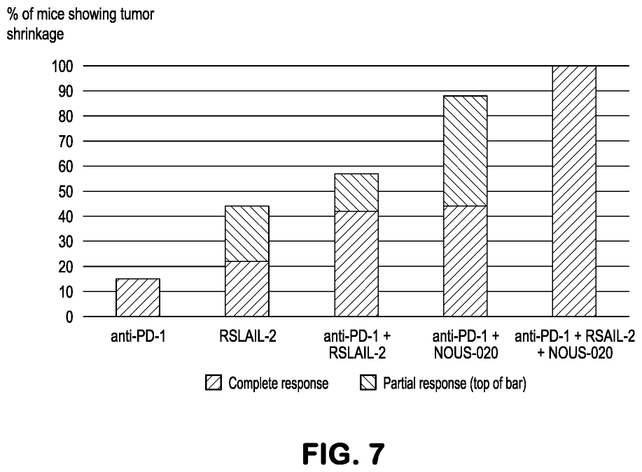

The first day of administration of any agent was day 0, except for RSLAIL-2 (d6) and MVA (Gr4 and Gr5) delivered at day 30. RSLAIL-2 (0.8 mg/kg) was administered three times, every 8-9 days between injections until the end of the study. Anti-PD1 was administered twice a week for 2 weeks, 200 μg i.p. NOUS-020 GAd vaccine (5×10⁸ vp) was injected once im at day 0, MVA (10⁷ pfu) at day 30 im. Tumor volume was measured over time. CR=complete response (tumor free mice) and PR=partial response (>40% tumor shrinkage). Results are shown in FIG. 7.

As shown in FIG. 7, therapeutic treatment with a combination of NOUS-020 vaccine (GAd and MVA), RSLAIL-2 and anti-PD-1 strikingly resulted in complete regression of established tumors in all treated animals, demonstrating the unexpected superiority of this combination modality.

1. A method of treating a subject having cancer comprising administering to the subject,
(i) a neoantigen-based vaccine composition comprising a first vector comprising a nucleic acid construct encoding multiple immunogenic polypeptide fragments, each of a protein mutated in cancer cells, wherein each immunogenic polypeptide fragment comprises one or more mutated amino acids flanked by a variable number of wild type amino acids from the original protein, joined head-to-tail to form an immunogenic polypeptide, and
(ii) a long acting, IL-2Rβ-selective agonist composition comprising compounds of Formula (I) 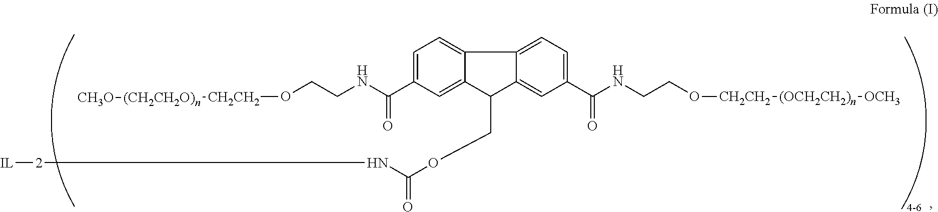
wherein IL-2 is interleukin-2, “—NH-IL-2” represents an amino group of IL-2, and each integer (n) has a value from about 200-300, or pharmaceutically acceptable salt forms thereof.

2. The method of claim 1 wherein the lengths of each of the immunogenic polypeptide fragments vary.

3. The method of claim 1 or claim 2, wherein the nucleic acid construct encodes an immunogenic polypeptide comprising (i) at least 1 immunogenic polypeptide fragment, or (ii) at least 4 immunogenic polypeptide fragments, or (iii) more than 19 polypeptide fragments, each of a protein mutated in cancer cells, joined head-to-tail.

4. The method of claim 3, wherein the nucleic acid construct encodes an immunogenic polypeptide comprising more than 19 polypeptide fragments, each of a protein mutated in cancer cells, joined head-to-tail.

5. The method of claim 1, wherein the vector is an adenoviral vector.

6. The method of claim 5, wherein the adenoviral vector is a nonhuman great ape-derived adenoviral (GAd) vector.

7. The method of any one of claims 1-6, wherein the a neoantigen-based vaccine composition further comprises a second vector comprising a nucleic acid construct encoding multiple immunogenic polypeptide fragments, each of a protein mutated in cancer cells, wherein each immunogenic polypeptide fragment comprises one or more mutated amino acids flanked by a variable number of wild type amino acids from the original protein, joined head-to-tail to form an immunogenic polypeptide, and at least one epitope of the immunogenic polypeptide encoded by the first vector is immunologically identical to at least one epitope of the immunogenic polypeptide encoded by the nucleic acid construct of the second vector, for use in a prime-boost vaccination regimen.

8. The method of claim 7, wherein the second vector is a modified vaccinia Ankara (MVA) vector.

9. The method of claim 8, wherein the second vector comprises a nucleic acid construct encoding multiple immunogenic polypeptide fragments, each of a protein mutated in cancer cells, and the multiple polypeptide fragments comprise at least 4 immunogenic polypeptide fragments, or more than 19 polypeptide fragments, each of a protein mutated in cancer cells, joined head-to-tail.

10. The method of any one of claims 1-9, wherein the long acting, IL-2Rβ-selective agonist composition contains no more than about 10 percent (molar) of compounds encompassed by the following formula: 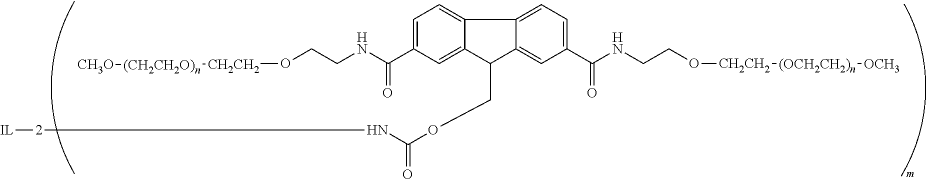
wherein (m) is an integer selected from the group consisting of 1, 2, 3, 7 and >7, or
pharmaceutically acceptable salt forms thereof, and each integer (n) has a value from about 200-300.

11. The method of any one of claims 1-10, wherein each branched polyethylene glycol of Formula (I) has a weight average molecular weight of about 20,000 daltons.

12. The method of any one or more of the foregoing claims, wherein the cancer is selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, Hodgkin's disease and adrenocortical cancer.

13. The method of any one of claims 1-12, wherein the neoantigen-based vaccine composition and the long acting, IL-2Rβ-selective agonist composition are initially administered sequentially, in any order.

14. The method of claim 13, wherein the neoantigen-based vaccine composition is administered to the subject prior to administering the long acting, IL-2Rβ-selective agonist composition.

15. The method of any one of claims 1-13, wherein the neoantigen-based vaccine composition and the long acting IL-2Rβ-selective agonist composition are both administered on day 1 of treatment.

16. The method of claim 14, wherein the neoantigen-based vaccine composition is administered on day 1 of treatment and the long-acting IL-2Rβ-selective agonist composition is administered on a day greater than 5 days following vaccination (e.g., on day 6, 7, 8, 9, 10, 11, 12, 13, 14, or 15 of treatment).

17. The method of any one of the foregoing claims, comprising a first administering of the neoantigen-based vaccine composition for priming an immune response in the subject and a subsequent administration of the same vaccine composition or a vaccine composition directed against the same disease for boosting the immune response in the subject.

18. The method of claim 17, wherein the subsequent administration for boosting the immune response of the subject is administered within 1 week, 2 weeks, 3 weeks or 4 weeks or more following the first administering.

19. The method of any one or more of the preceding claims, wherein following a first cycle of treatment comprising administration of at least the neoantigen-based vaccine composition and the long acting IL-2Rβ-selective agonist composition, additional cycles of treatment comprising administration of the long-acting IL-2Rβ-selective agonist composition are carried out.

20. The method of any of the foregoing claims, wherein the subject is a human.

21. The method of any one of the foregoing claims, wherein the cancer is a solid cancer.

22. The method of any one of the preceding claims, wherein the dose of the long-acting IL-2Rβ-selective agonist composition ranges from about 0.0001 mg/kg to about 0.1 mg/kg body weight.

23. The method of any one or more of the preceding claims, further comprising administering to the subject an anti-PD-1 antibody.

24. The method of claim 23, wherein the anti-PD-1 antibody, preferably nivolumab, is administered (i) on the same day as the a neoantigen-based vaccine composition, or (ii) on the same day as the long-acting IL-2Rβ-selective agonist composition, or (iii) following administering of the a neoantigen-based vaccine composition, or (iv) following administering of the long-acting IL-2Rβ-selective agonist composition, or (v) following administering of the a neoantigen-based vaccine composition but before administering of the long-acting IL-2Rβ-selective agonist composition.

25. The method of claim 23 or claim 24, wherein the anti-PD-1 antibody, preferably nivolumab, is administered more than once over the course of therapy.

26. The method of any one of claims 23-25, wherein the neoantigen-based vaccine composition, the long-acting IL-2Rβ-selective agonist composition, and the anti-PD-1 antibody, preferably nivolumab, are each administered as separate compositions.

27. The method of any one of claims 1-25, wherein the cancer comprises a cancerous tumor and the method is effective to reduce the size of the cancerous tumor when compared to the size of the tumor prior to the administering.

28. The method of any one of claims 23-27, wherein the cancer comprises a cancerous tumor and the method is effective to reduce the size of the cancerous tumor by at least about 30% when compared to the size of the tumor prior to the administering.

29. The method of claim 28, wherein the cancer comprises a cancerous tumor and the method is effective to reduce the size of the cancerous tumor by at least about 90% or more when compared to the size of the tumor prior to the administering.

30. The method of claim 29, wherein the cancer comprises a cancerous tumor and the method is effective to result in complete tumor regression.
